(19) United States
(12) Patent Application Publication
(10) Pub. No.: US 2025/0387269 A1
(43) Pub. Date: Dec. 25, 2025

(54) WOUND DRESSING WITH SENSOR

(71) Applicant: CONVATEC LIMITED, Flintshire (GB)

(72) Inventors: Jade Steven, Deeside (GB); Brian Murphy, Flintshire (GB)

(21) Appl. No.: 19/305,731

(22) Filed: Aug. 20, 2025

Related U.S. Application Data
(63) Continuation of application No. PCT/GB2024/050440, filed on Feb. 19, 2024.
(60) Provisional application No. 63/446,982, filed on Feb. 20, 2023.

(51) Int. Cl. A61F 13/00; A61F 13/0203

(52) U.S. Cl. CPC A61F 13/00055; A61F 13/022; A61F 2013/00182

(57) ABSTRACT

An exemplary wound dressing generally includes a wound-contacting layer and a sensor array. The sensor array is positioned distally of the wound-contacting layer, and generally includes a substrate, a first sensor positioned on the substrate, and a second sensor positioned on the substrate. The second sensor surrounds the first sensor, and each of the first sensor and the second sensor has a corresponding and respective electrical characteristic that varies in response to contact with wound exudate.

### CROSS-REFERENCE TO RELATED APPLICATIONS

The present application claims the benefit of U.S. Provisional Patent Application No. 63/446,982, filed 20 Feb. 2023, the contents of which are incorporated by reference in their entirety.

### TECHNICAL FIELD

The present disclosure generally relates to wound dressings, and more particularly but not exclusively relates to systems and methods for determining the appropriate time to change a wound dressing.

### BACKGROUND

In chronic wounds, exudate management can play a critical role, as the exudate produced is considered to be a corrosive biological fluid due to its range of harmful constituents (e.g. bacteria and enzymes). When healthy peri-wound skin is exposed to wound exudate, it can become macerated and excoriated, and potentially lead to peri-wound moisture-associated skin damage (MASD). This moisture-associated skin damage can further breakdown the peri-wound skin, which may disrupt the healing process and lead to an increase of wound tissue.

The effective management of wound exudate and the importance of reducing the exposure to harmful constituents can be an important aspect of protecting the healing tissue and in helping prevent further tissue breakdown.

The changing of wound dressings by health care professionals (HCPs) is not an exact science, and is usually dependent on a visual inspection of the wound and dressing. Since most wound patients are treated in the community, predominantly by community and generalist nurses, this challenge is exacerbated by the relatively low levels of specific wound training in many healthcare settings. The result can be a debilitating personal cost to the patient, and a draining economic cost to healthcare systems. Traditionally, visual inspections will rely on the clinician removing the dressing; this can lead to the removal of the dressing being premature, accurate, or overdue. Outdated behaviors and strict adherence to established but dated protocols may result in dressings being changed more frequently than necessary, which can add to inefficiencies and increasing risk of further wound complications. Undisturbed wound healing is the benchmark for optimizing outcomes whilst improving quality of care for patients. However, many conventional dressings leave these needs unattended. For these reasons among others, there remains a need for further improvements in this technological field.

### SUMMARY

An exemplary wound dressing generally includes a wound-contacting layer and a sensor array. The sensor array generally includes a substrate, a first sensor positioned on the substrate, and a second sensor positioned on the substrate. The second sensor surrounds the first sensor, and each of the first sensor and the second sensor has a corresponding and respective electrical characteristic that varies in response to contact with wound exudate. Further embodiments, forms, features, and aspects of the present application shall become apparent from the description and figures provided herewith.

### BRIEF DESCRIPTION OF THE FIGURES

FIG. 1 is an exploded view of a wound dressing according to certain embodiments.

FIG. 2 is a plan view of a sensor array according to certain embodiments.

FIG. 3 is a graph illustrating observed current versus exposed length in a sensor array according to certain embodiments.

FIG. 4 is a graph illustrating open current voltage (OCV) and closed-circuit current (CCI) versus time for a sensor array according to certain embodiments.

FIG. 5 is a plan view of a sensor array according to certain embodiments.

FIG. 6 is a schematic diagram of a control circuit according to certain embodiments.

FIG. 7 is a graph illustrating signal versus time for a sensor array according to certain embodiments.

FIG. 8 is an enlarged portion of the graph illustrated in FIG. 7, both before correction (above) and after correction (below).

FIG. 9 is a graph illustrating signal versus time for a sensor array according to certain embodiments.

FIG. 10 is a graph illustrating signal versus time for a sensor array according to certain embodiments.

FIG. 11 is a schematic diagram of a control circuit according to certain embodiments.

FIG. 12 is a graph illustrating signal versus time during implementation of a debias cycle according to certain embodiments.

FIG. 13A is a graph illustrating signal versus time for a sensor array utilizing carbon.

FIG. 13B is a graph illustrating signal versus time for a sensor array utilizing silver.

FIG. 14 is a schematic diagram of a control circuit according to certain embodiments.

FIG. 15 is a graph illustrating voltage versus time for a test of a sensor array according to certain embodiments.

FIG. 16 is a plan view of a sensor array according to certain embodiments.

FIG. 17 is a schematic representation of a stage of an example assembly process.

FIG. 18 is a cross-sectional view of a portion of a wound dressing assembly according to certain embodiments.

FIG. 19 is a schematic representation of a test configuration for a sensor array.

FIG. 20 is a graph illustrating signals versus time for a test of a sensor array according to certain embodiments.

FIG.21(a) through FIG.21(e) illustrate the extent of liquid absorption at various stages during a test of a sensor array.

FIG. 22 is a schematic block diagram of a system according to certain embodiments.

FIG. 23 is a schematic block diagram of a computing device that may be utilized in connection with certain embodiments.

FIG. 24 is a schematic block diagram illustrating a control circuit according to certain embodiments.

FIG. 25 is a graph illustrating voltages during operation of an illustrative implementation of the control circuit illustrated in FIG. 24.

FIG. 26 is a first graph illustrating signals during an example test of the control circuit illustrated in FIG. 24.

FIG. 27 is a second graph illustrating signals during an example test of the control circuit illustrated in FIG. 24.

FIG. 28 is a graph illustrating operation of the control circuit per a process according to certain embodiments.

FIG. 29 illustrates output of a sensor array for a dry sensor (FIG. 29A), a mildly-wetted sensor (FIG. 29B) and a highly-wetted sensor (FIG. 29C).

FIG. 30 is a schematic flow diagram of a process according to certain embodiments.

FIG. 31 is a schematic time-flow diagram for a process according to certain embodiments.

FIG. 32 illustrates various electrical characteristics during a test according to certain embodiments.

### DETAILED DESCRIPTION OF ILLUSTRATIVE EMBODIMENTS

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should further be appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described.

As used herein, the terms “proximal” and “distal” may be used to denote two opposite directions relative to a wound site. More particularly, the term “proximal” may be used to indicate a direction that extends toward the wound site, and the term “distal” may be used to indicate a direction that extends away from the wound site. Thus, in certain wound dressings described herein, the wound contacting layer is a more-proximal layer, and a backing layer is a more-distal layer.

Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Items listed in the form of “A, B, and/or C” can also mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

In the drawings, some structural or method features may be shown in certain specific arrangements and/or orderings. However, it should be appreciated that such specific arrangements and/or orderings may not necessarily be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may be omitted or may be combined with other features.

The disclosed embodiments may, in some cases, be implemented in hardware, firmware, software, or a combination thereof. The disclosed embodiments may also be implemented as instructions carried by or stored on one or more transitory or non-transitory machine-readable (e.g., computer-readable) storage media, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

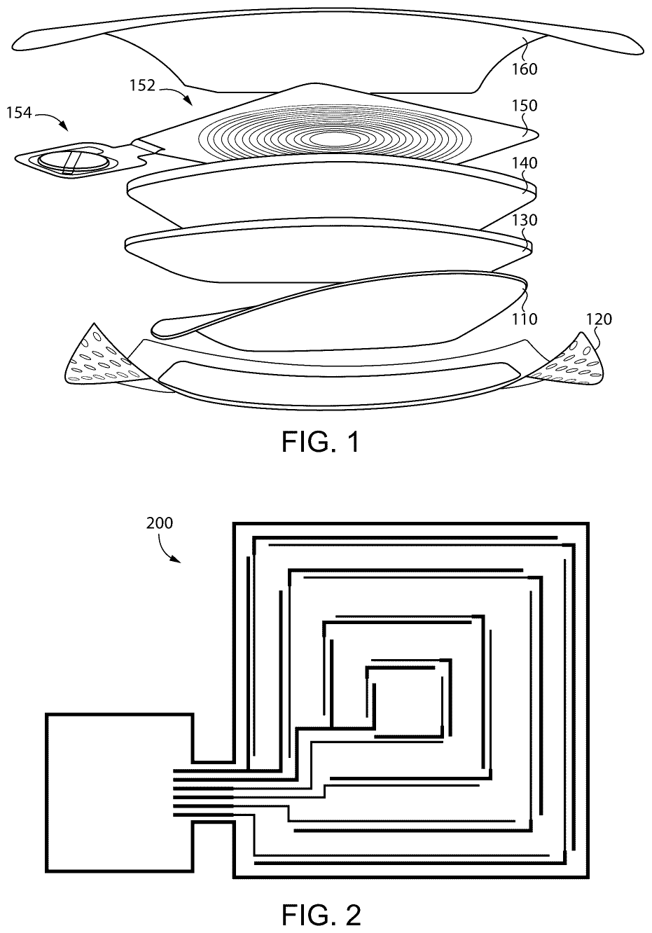

With reference to FIG. 1, illustrated therein is a wound dressing 100 according to certain embodiments. The wound dressing 100 generally includes a wound-contacting layer 110, an adhesive layer 120 at least partially surrounding the wound-contacting layer 110, a foam layer 130 positioned distally of the wound-contacting layer 110, an absorbent layer 140 positioned distally of the foam layer 130, a sensing layer 150 positioned distally of the sensing absorbent layer 140, and a backing layer 160 positioned distally of the sensing layer 150.

The wound-contacting layer 110 is configured to contact the wound and to absorb exudate from the wound. In certain embodiments, the wound-contacting layer 110 may be a gelling layer. For example, the wound-contacting layer 110 may include woven or non-woven fibers that, when contacted by exudate, turn into a gel. In certain forms, the wound-contacting layer may, for example, be formed of Hydrofiber®.

The adhesive layer 120 aids in securing the wound dressing 100 to the wound and/or peri-wound skin, and at least partially surrounds the wound-contacting layer 110. The adhesive layer 120 may include a silicone adhesive. In certain embodiments, the adhesive layer 120 may comprise a silicone trilaminate material.

The foam layer 130 is positioned distally of the wound-contacting layer 110, and is configured to retain at least some moisture while transmitting excess moisture to the absorbent layer 140. In certain embodiments, the foam layer 130 may be a polyurethane foam layer.

The absorbent layer 140 is positioned distally of the wound-contacting layer 110, and in the illustrated form is positioned distally of the foam layer 130. As described herein, the absorbent layer 140 is configured to absorb exudates from the wound. In certain embodiments, the absorbent layer 140 may be a superabsorbent layer.

The sensing layer 150 is positioned distally of the absorbent layer 140, and generally includes a sensor array 152 operable to detect moisture in the absorbent layer 140. In certain embodiments, the sensing layer 150 may further include a printed circuit board assembly (PCBA) 154 that aids the sensor array 152 in generating its output and/or in communicating the information generated by the sensor array 152 to an external device.

The backing layer 160 is positioned distally of the sensing layer 150, and aids in preventing exudate from passing fully through the wound dressing 100. The backing layer 160 may, for example, be formed of a polyurethane film.

Designing a dressing with an embedded microelectronic sensor array 152 involves consideration of many factors, such as detecting and monitoring the variables related to the absorption of wound exudate (e.g., ratio of wet area to dry area, and when the dressing should be renewed). One potential use of the sensor array 152 is to monitor the proportion of exudate absorbed. As a result, the location of the sensor array 152 can be significant, and may typically be the furthest distance from the wound bed. For example, in the embodiment illustrated in FIG. 1, the sensing layer 150 is proximal only to the backing layer 160. The configuration and/or pattern of the sensor array 150 may involve multi-zone detection in order to provide the user with incremental updates on the percentage of wet area.

In determining the sensing technology to be utilized in the sensing layer 150, one may consider factors such as accuracy, disturbance from external stimuli (e.g., pressure), complexity, energy consumption, cost, and/or other factors. Possible sensing technologies include, but are not limited to: electrochemical sensing technologies, resistive sensing technologies, thermal sensing technologies, galvanic sensing technologies, and capacitive sensing technologies. Although thermal sensing has the advantage of multizone sensing, thermal sensors may require positive temperature coefficient (PTC) ink coupled with a complex processing algorithm. While galvanic sensing has the benefit of producing a strong signal for up to 48 hours, after this time the variability of the signal and its internal resistance become difficult to characterize. Additionally, while capacitive sensing has the advantage that it does not require physical contact with moisture, these technologies typically have high power consumption. Thus, although thermal sensing, galvanic sensing, and capacitive sensing may be utilized in certain embodiments, electrochemical and/or resistive sensing may be more fruitful avenues to pursue.

With additional reference to FIG. 2, illustrated therein is an electrochemical sensor array 200 according to certain embodiments. The sensor array 200 may, for example, be utilized in the sensor array 152. The sensor array 200 pairs carbon and silver ink to create a battery. This relatively simple design has the benefit of limited sensitivity to applied external pressure, which may reduce false readings. As an electrochemical sensor typically requires contact with electrolyte-containing moisture, the sensor array 200 will typically be embedded within the wound dressing 100.

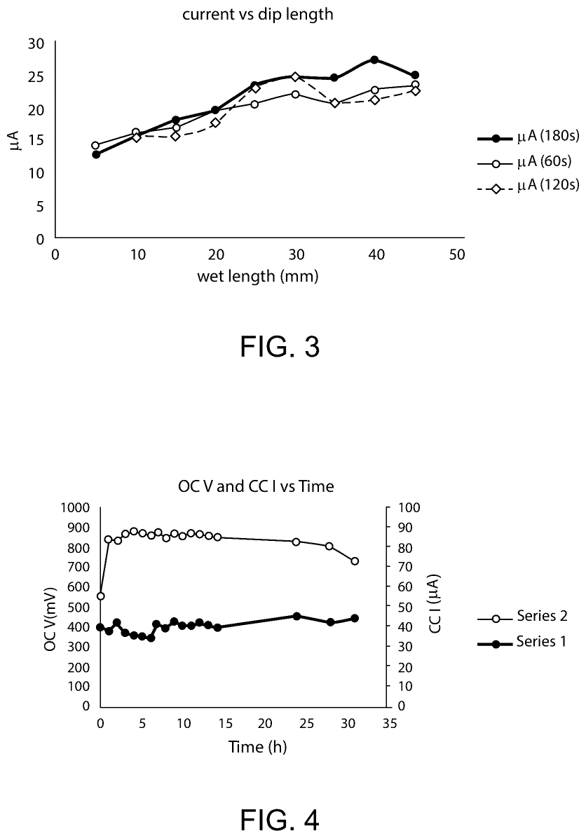

With additional reference to FIG. 3, preliminary testing of the electrochemical sensor array 200 (with a silver/zinc ink printed on a substrate) exposed to 0.9% sodium chloride solution verified the presence of proportionality between electrical current and wet ink length.

With additional reference to FIG. 4, over time, the measured open circuit voltage (OCV) and closed circuit current (CCI) demonstrated a stable build-up of voltage in the range of 700 mV-900 mV. The voltage then begins to discharge in a manner similar to the behavior of a traditional battery.

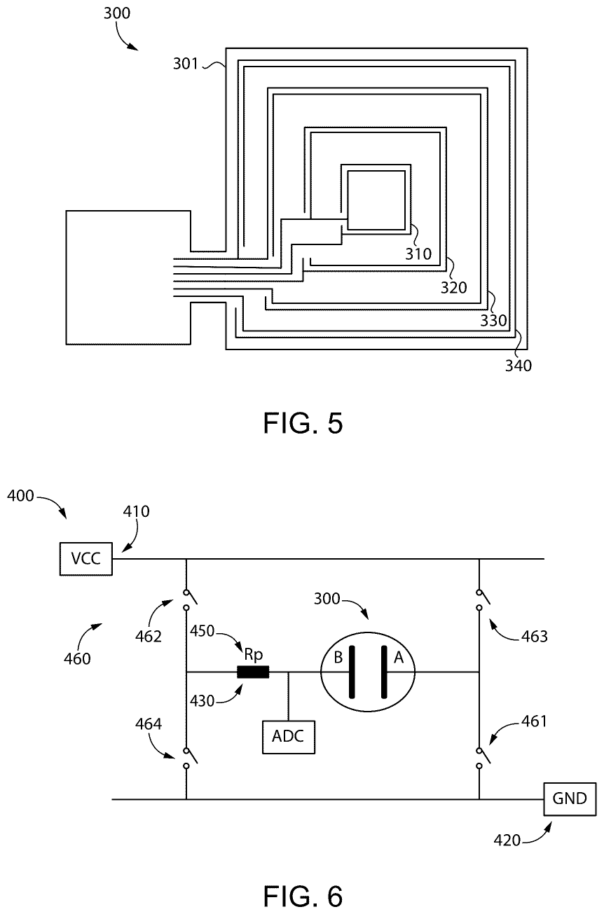

With additional reference to FIG. 5, illustrated therein is a resistive sensor array 300 according to certain embodiments. The sensor array 300 may, for example, be utilized as the sensor array 152 of the wound dressing 100. The resistive sensor array 300 includes a plurality of nested traces, including an innermost or first trace 310, and a second trace 320 surrounding the first trace 310 such that the first trace 310 is nested within the second trace 320. The sensor array 300 may further include a third trace 330 surrounding the second trace 320 such that the second trace 320 is nested within the third trace 330. In the illustrated form, the sensor array 300 further includes a fourth trace 340 surrounding the third trace 330 such that the third trace 330 is nested within the fourth trace 340. While the illustrated sensor array 300 includes four nested traces 310, 320, 330, 340, it is also contemplated that a sensor array according to other embodiments may include more or fewer traces. In certain embodiments, the traces may be considered to be sensing elements or sensors of the sensor array 300. The illustrated traces may be printed by an ink on a substrate 301. In certain embodiments, the ink may comprise silver. Preliminary testing of resistive sensors using silver traces have shown positive multi-zone sensing. Stated another way, each trace 310, 320, 330, 340 can produce an independent reading, and can thus function as an independent sensor of the sensor array 300.

In dry conditions, the full open circuit of the sensor array 300 can register at greater than 10 MΩ. It has been found that in the presence of an electrolyte solution, such as a 0.9% sodium chloride solution, the registered range is about 1 to 2 MΩ. While resistivity is maintained in the four independent traces, it has been found that the reading may be inaccurate and/or unstable. Using direct current (DC) test conditions, the silver ink of the sensor oxidizes and therefore results in bias over time, and creates a chemical cross-talk effect from adjacent traces. To overcome the bias over time, it may be necessary to use an algorithm with a depolarization cycle before the true measurement is taken and hence adopt an impedentiometric approach instead of resistive.

With additional reference to FIG. 6, in order to measure impedance, a new alternating current (AC) driving circuit 400 may be implemented. The circuit 400 generally includes a Voltage Common Corrector (VCC) line 410, a ground line 420, and an additional line 430 connected to an analog-digital converter (ADC) 440. Connected with the additional line 430 on opposite sides of the ADC 440 are a pull-up resistor (Rp) 450 and the sensor array 300, which includes an A side and a B side. The driving circuit 400 also includes a set of switches 460, including first through fourth switches 461-464.

In the first half-cycle of the driving scheme, Sensor Side A is connected to the ground line 420 via the closed first switch 461, Sensor Side B is connected to the VCC 410 via the pull-up resistor 450 and the closed second switch 462, and the third and fourth switches 463, 464 remain open. In the second half-cycle, Sensor Side A is connected to the VCC line 410 via the closed third switch 463, Sensor Side B is connected to the GND line 420 via the pull-down resistor 450 and the closed fourth switch, and the first and second switches 461, 462 remain open.

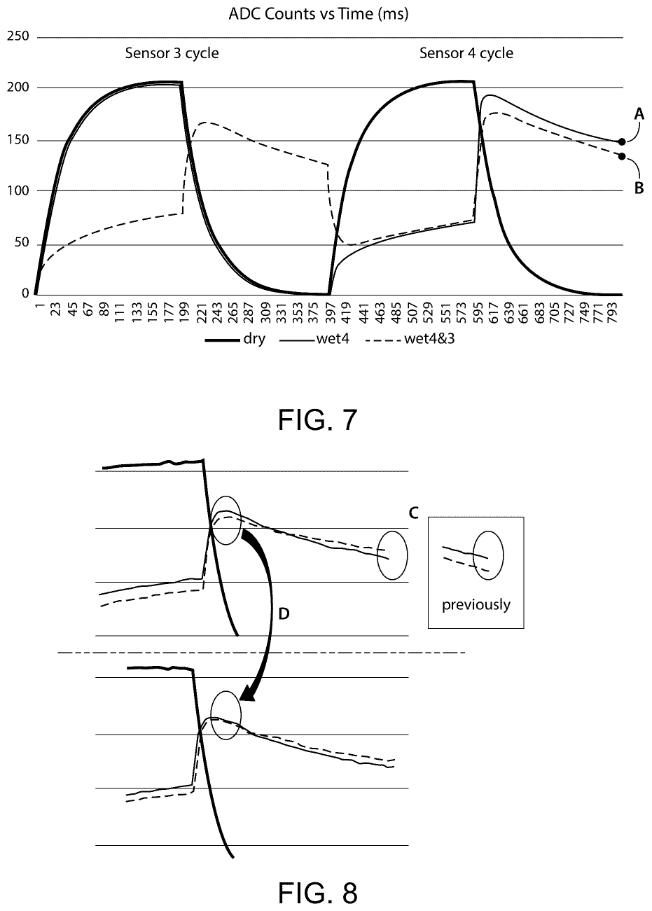

With additional reference to FIG. 7, illustrated therein is the chemical cross-talk effect. In FIG. 7, point A shows the Sensor4 wet readout when Sensor3 is dry, and point B shows the Sensor4 wet readout when Sensor3 is wet. Due to the sharing of a common electrode, the Sensor3 reading cycle is altering the response of Sensor4. This is apparent as the Sensor4 wet readout is lower than the wet Sensor3 readout.

With additional reference to FIG. 8, illustrated therein are similar graphs as shown in FIG. 7 after correction. Using the AC driving scheme 400, the pull-up was reduced from 300 k to 47 k, and the bias time was reduced from 200 ms to 50 ms. The Sensor4 reading increased to be higher when Sensor3 is simultaneously wet. By adding a delay between channel sampling (e.g., a five-second delay), a reduction of the chemical crosstalk can be achieved. FIG. 8 illustrates the wet Sensor4 reading after changes to bias time (indicated with C) and chemical crosstalk (indicated with D) with simultaneous wet Sensor3.

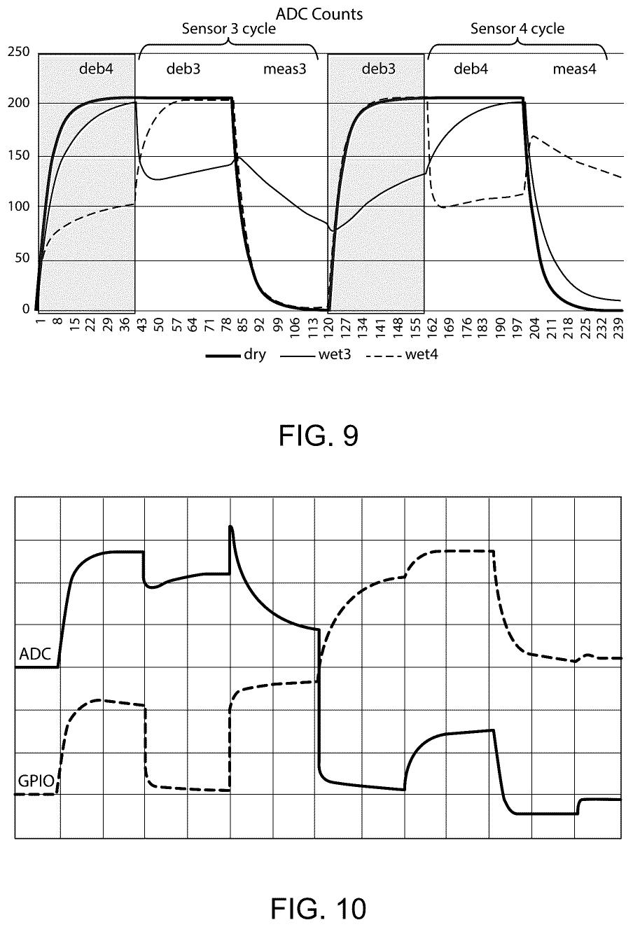

With additional reference to FIG. 9, in order to further reduce the chemical crosstalk, a de-bias cycle may be introduced on the adjacent channel prior to the measurement cycle.

With additional reference to FIG. 10, due to the capacitance build up coming from the electrolyte-bearing moisture, when the sensor track is depolarised, the General Purpose Input/Output (GPIO) line (left in three state) is bounced below ground potential-resulting in an offset reading of the next channel and causing a diode clamping effect.

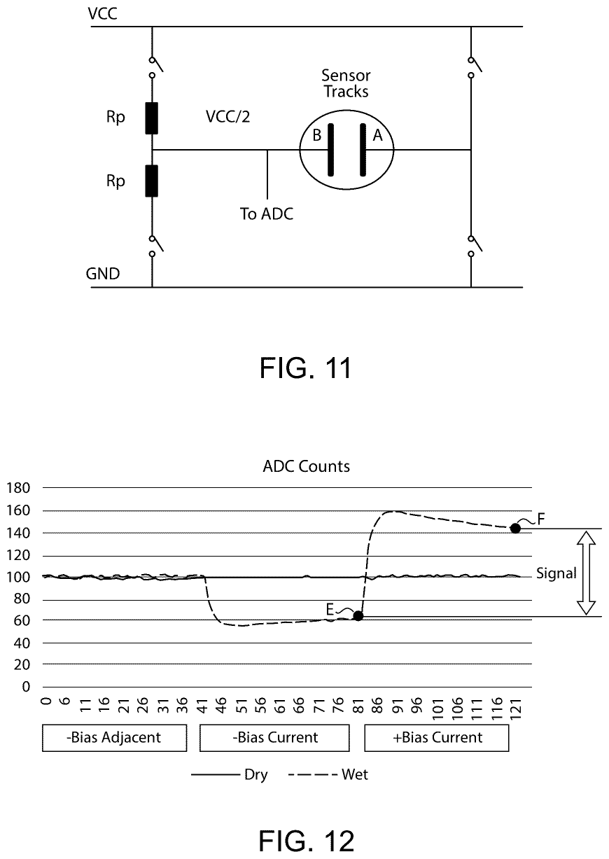

With additional reference to FIGS. 11 and 12, in order to prevent diode activation, the biasing scheme was modified to use pull up resistors to VCC/2 and switch the other side of the cell to VCC and GND, thereby resulting in a +VCC/2 and −VCC/2 bias as shown in FIG. 11. The pull-up was set to 47 k to VCC and 57 k to GND with a timing of 40 ms. A differential algorithm was implemented, and the difference between Point E and Point F was interpreted as the resulting signal, as shown in FIG. 12.

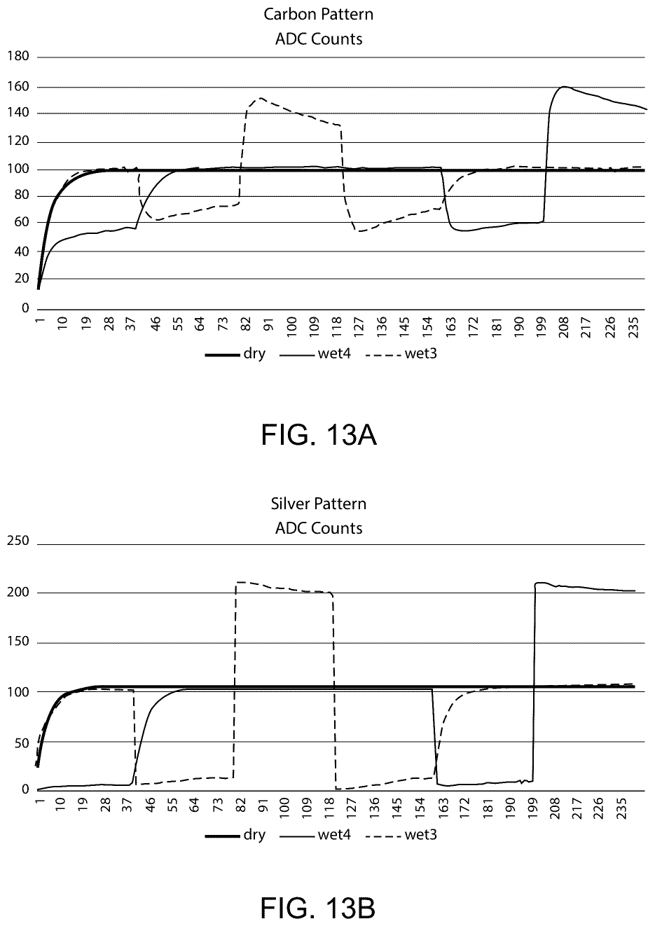

With additional reference to FIGS. 13A and 13B, the enhanced biasing scheme was tested with a carbon trace pattern (FIG. 13A) and a silver trace pattern (FIG. 13B). The measurement was improved and no major chemical crosstalk or diode activation was observed.

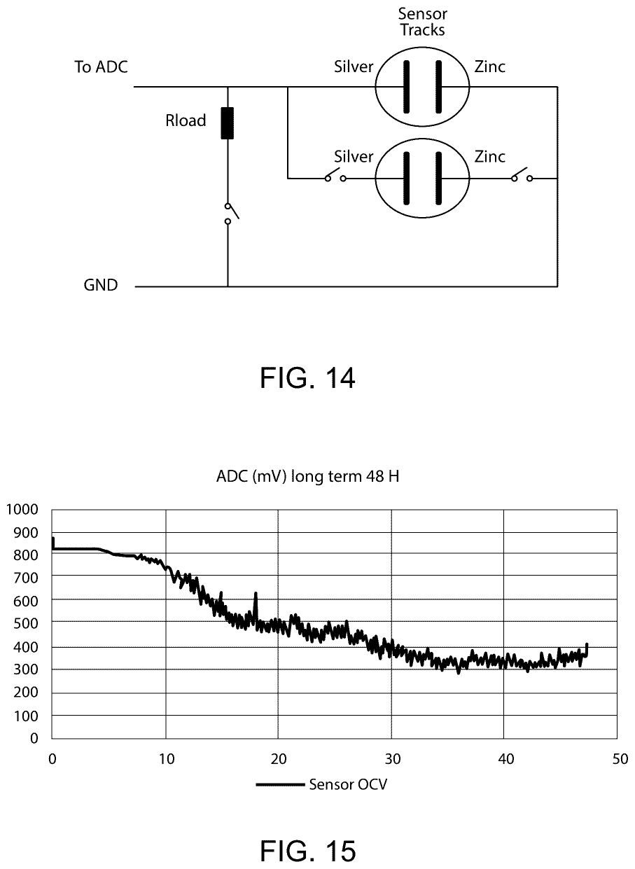

With additional reference to FIG. 14, illustrated therein is a galvanic sensor with two silver/zinc sensor tracks. The sensor is configured with a resistive load (R_{load}). In order to measure galvanic response, two strips of substrate with silver/zinc printed inks were exposed to a 0.9% sodium chloride solution. The galvanic sensor is arranged in a dressing construct as per FIG. 1, and the sensor connections are displayed in FIG. 14.

With additional reference to FIG. 15, the long term signal behavior was then measured over 48 hours with the sensor in contact with an absorbent dressing. It was found that the ADC begins to decline after approximately eight hours, and the signal continues to degrade over time.

The signals and lifetime of the sensor depend in part upon the chemical composition of the liquid. Due to the variability of the signal and its internal resistance over time after prolonged exposure, it is difficult to characterize the behavior of a wet trace, since different conditions are present on the same trace.

The configuration/layout of the dressing 100, as shown in FIG. 1, places a gelling fiber layer (e.g., Hydrofiber® or another gelling fiber) as the wound contact layer 110, which is furthest from the sensing layer 150. It has been found that the behavior of moisture within the dressing is anisotropic, and that the absorbed fluid follows the path of least resistance through the different layers. Taking into consideration the anisotropic nature of the dressing 100, the pattern, quantity, and shape of the sensor(s) may be optimized. In certain embodiments, the sensor pattern is resilient to non-uniform wetting patterns due to wound placement, dressing anisotropy, gravity, external pressure and movement. Shorter traces in a pattern will allow for smaller series resistance and overall better signal quality. The pattern should also be able to detect moisture in the corners of the dressing to provide a complete view of the wet area.

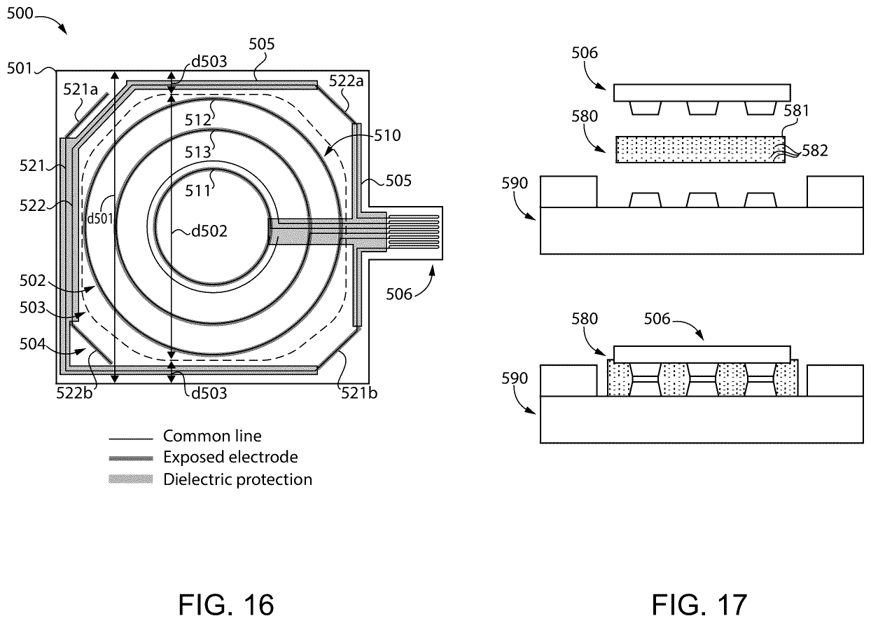

With additional reference to FIG. 16, illustrated therein is a sensor array 500 according to certain embodiments. The sensor array 500 may, for example, be utilized as the sensor array 152 of the wound dressing 100, and may be controlled according to the control schemes set forth herein. The sensor array 500 generally includes a substrate 501 and a plurality of sensor traces that may, for example, be printed on the substrate 501. The sensor traces include one or more interior traces 510 configured to sense moisture in various zones within the interior region 502 of the substrate 501, and/or one or more border traces 520 configured to sense moisture within the border region 503 of the substrate 501, for example at the corner regions 504 of the substrate 501. The sensor array 500 may include a protection layer 505 configured to isolate selected portions of the traces from exposure to exudate. The sensor array 500 may include a bridge 506 configured for connection to an external device operable to control the sensor array 500 and/or to receive signals transmitted by the sensor array 500.

The illustrated sensor array 500 includes one or more interior traces 510, including an innermost first interior trace 511. In the illustrated form, the sensor array 500 includes a plurality of interior traces 510, the plurality of interior traces 510 further including a second interior trace 512 surrounding the first interior trace 511 such that the first interior trace 511 is nested within the second interior trace 512. The sensor array 500 may further include a third interior trace 513 surrounding the second interior trace 512 such that the second interior trace 512 is nested within the third interior trace 513. In certain embodiments, portions of the traces leading from the exposed regions to the bridge 506 may be coated with a protection layer 505 to isolate the lead and return lines from exposure to exudate. While the illustrated sensor array 500 includes three nested interior traces 510, it is also contemplated that the sensor array 500 may include more or fewer interior traces, and that the interior traces 510 may not necessarily be nested within one another. Moreover, while the illustrated interior traces 510 are generally circular, it should be appreciated that interior traces according to other embodiments may have different geometries.

In the illustrated form, the traces are provided as nested traces, in which each trace surrounds and/or is surrounded by at least one other trace. It is also contemplated that the sensor array 500 may have a different configuration, such as one in which the traces are not nested. By way of example, one or more traces may instead be formed in a grid-like pattern.

As noted above, the illustrated sensor array 500 includes one or more border traces 520 configured to sense moisture in the border region 503. In certain embodiments, the border region 503 may have a dimension d503 that has a predetermined relationship with a corresponding dimension d501 of the substrate 501 and/or a corresponding dimension d502 of the interior region 502. In certain embodiments, the border region dimension d503 may be 5% or less of the substrate dimension d501 such that the interior region dimension d502 is 90% or more of the substrate dimension d501. In certain embodiments, the border region dimension d503 may be 10% or less of the substrate dimension d501 such that the interior region dimension d502 is 80% or more of the substrate dimension d501. In certain embodiments, the border region dimension d503 may be 15% or less of the substrate dimension d501 such that the interior region dimension d502 is 70% or more of the substrate dimension d501.

In the illustrated form, the one or more border traces 520 includes a first border trace 521 and a second border trace 522, each of which is configured to sense moisture in a corresponding pair of the corner regions 504. In the illustrated form, the first border trace 521 includes two exposed regions 521a, 521b positioned on two opposite corners 504, and the second border trace 522 includes two exposed regions 522a, 522b positioned on the other two corners 504. The remainder of the border traces 521, 522 (i.e., the portions other than the exposed regions 521a, 521b, 522a, 522b) may be coated with a protection layer 505 such that the non-exposed regions are isolated from contact with exudate.

As should be appreciated from the foregoing, the illustrated sensor pattern features three generally circular electrodes for radial detection of exudate, and two pairs of corner sensors for detection of exudate in the corner regions 504, thereby providing coverage of the dressing area. The complexity of the dielectric protection 505 on the sensor traces may impact the breathability of the sensor array 500. A moist wound healing environment is conducive for better healing outcomes, and as such the breathability and moisture vapor transfer through the backing layer 160 may need to be considered.

With additional reference to FIG. 17, anisotropic conductive film (ACF) 580 is a composite material made by an adhesive 581 having conductive particles 582 carried therein. It may be protected by a shielding layer and removed during application. The ACF 580 is compressed between PCB pads 590 (e.g., of the PCBA 154) and the sensor array 500, and the conductive particles 582 allow for electrical continuity between the pads 590 and the conductive traces.

In certain embodiments, the sensor substrate layer 501 can be transparent. By way of example, the substrate 501 may be formed of Lubrizol® FSL85B4P or another suitable transparent substrate. While transparent substrates may be preferred in certain embodiments (such as when easier visual inspection is desired), it is also contemplated that the substrate layer 501 may be formed of an opaque material, such as Bemis® ST604 or another suitable opaque substrate. In certain embodiments, the ink utilized to print the traces may be a silver ink, which shows a better interaction with ACF material as compared with carbon based inks.

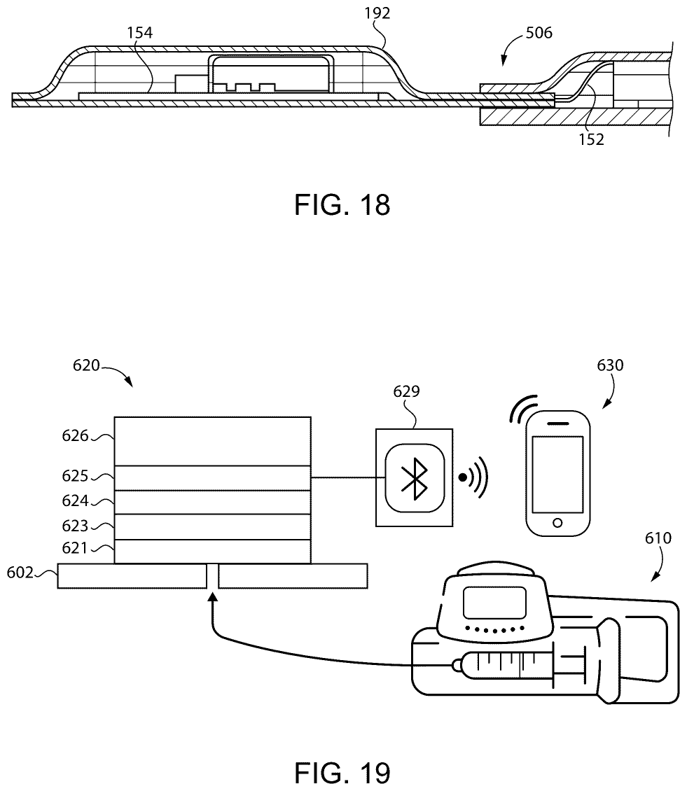

With additional reference to FIG. 18, the PCBA 154 and the sensor bridge 506 may need to be protected from moisture and external forces (e.g., pressure and cracking). One option for protecting the sensor bridge 506 involves embedding the PCBA 154 into foam 192, and sandwiching the PCBA 154 and bridge 506 between foam 192 laminated with thermoplastic polyurethane film and sealed via heat lamination.

In certain embodiments, the subject matter disclosed herein may be utilized in a wound care setting in order to convey to a user the level of wetness within the dressing. In order to do so, the PCBA 154 may need to communicate with an external device that logs the information generated by the sensor array 152.

With additional reference to FIG. 19, illustrated therein is an example test configuration 600 for a sensor array such as the sensor array 500. The test configuration generally includes an infusion pump 610, a test dressing 620, and an external device 630. As described herein, the infusion pump 610 delivers liquid at a predetermined rate to the test dressing 620, which generates information for use by the external device 630.

During the test procedure, the infusion pump 610 delivers liquid at a predetermined rate to the test dressing 620 to emulate a weeping wound. For purposes of one test, the infusion pump 610 delivered an aqueous solution of 0.9% sodium chloride at a rate of 50 ml/hr to the test dressing 620.

The test dressing 620 is positioned on a support 602 through which the solution is delivered to the test dressing 620, which is substantially similar to the above-described wound dressing 100. The test dressing 620 generally includes a wound-contacting layer 621, a foam layer 623, an absorbent layer 624, and a sensing layer 625, which respectively correspond to the above-described wound-contacting layer 110, foam layer 130, absorbent layer 140, and sensing layer 150. In the illustrated embodiment, the test dressing 620 further includes a backing layer 626 covering the sensing layer 625. In order to improve visibility of the sensing layer 625, the backing layer 626 may be formed of a transparent material, such as a transparent acrylic sheet. While other embodiments are contemplated, in the illustrated form, the sensing layer 625 is provided in the form of the sensor array 500.

The test dressing 620 also includes a PCBA 629 corresponding to the PCBA 154. The PCBA 629 facilitates the control of the sensor array 500 and/or the transmission of sensor data to the external device 630. As described herein, the PCBA 629 may include a controller and/or a communication device. In the illustrated form, the communication device is a wireless communication device, and preferably a Bluetooth communication device, such as a Bluetooth Low Energy communication device. It is also contemplated that additional or alternative communication devices may be used, such as a wired communication device and/or an additional or alternative form of wireless communication device.

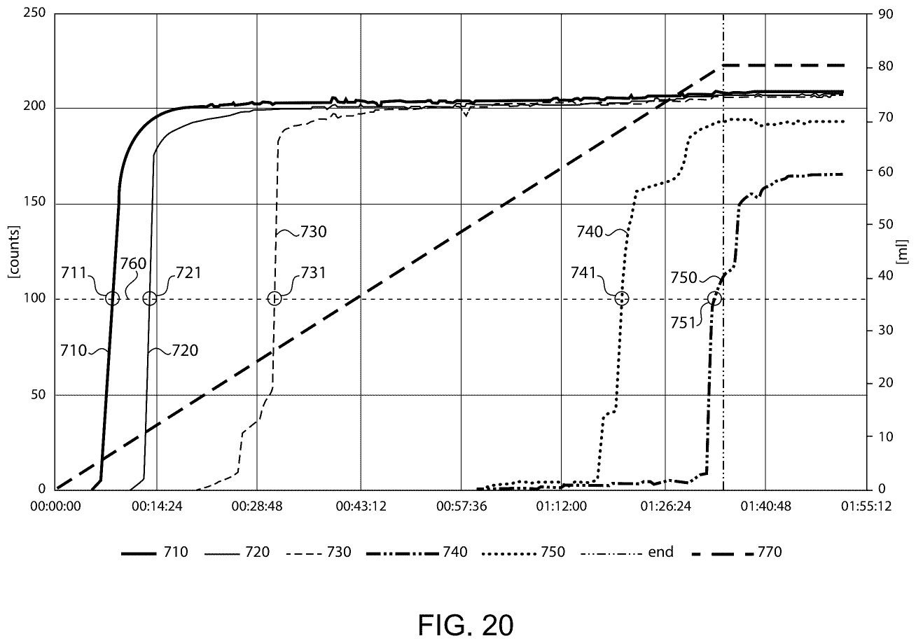

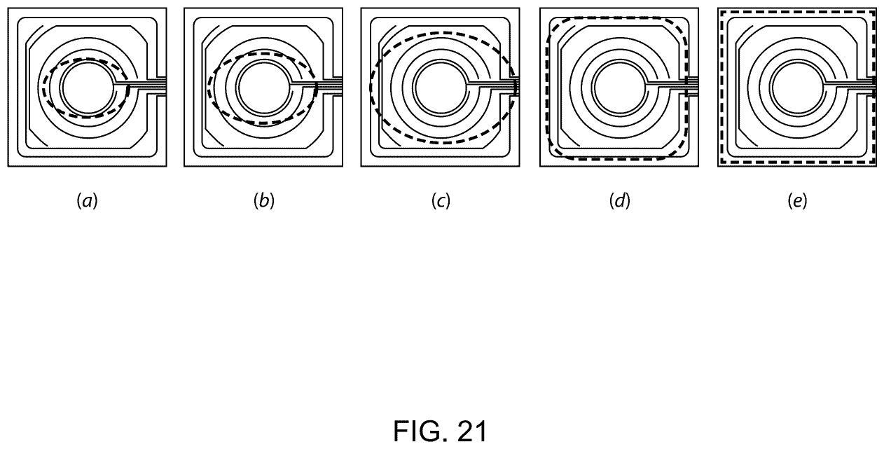

With additional reference to FIGS. 20 and 21, provided therein are certain results of a test performed using the test configuration 600 described above. More particularly, FIG. 20 is a graph 700 illustrating the outputs generated by the sensor array 500 during the test, and FIG. 21 illustrates the sensor array 500 at various stages during performance of the test.

Illustrated in the graph 700 of FIG. 20 are a first line 710 corresponding to the output of the first interior sensor 511, a second line 720 corresponding to the output of the second interior sensor 512, a third line 730 corresponding to the output of the third interior sensor 513, a fourth line 740 corresponding to the output of the first border sensor 521, and a fifth line 750 corresponding to the output of the second border sensor 522. As will be appreciated, the outputs illustrated in the first through fifth lines 710-750 may be generated according the procedures described herein. Also illustrated in the graph 700 is a sixth line 760 or threshold line 760 indicating the threshold selected for detection of wetness. The first through sixth lines 710-760 utilize the left Y-axis, which corresponds to the signals generated by the sensor array 500. While other units of measurement are contemplated, the illustrated left Y-axis corresponds to the number of counts generated by an analog-to-digital converter (ADC) of the PCBA 154/629.

Also illustrated in the graph 700 of FIG. 20 is a seventh line 770 corresponding to the amount of solution that the infusion pump 610 has delivered to the test dressing 620. The seventh line 770 utilizes the right Y-axis, which includes markers for the number of milliliters delivered to the test dressing 620.

The first line 710 (corresponding to the output of the first interior sensor 511) intersects the threshold line 760 at point 711, which indicates that the first interior sensor 511 was triggered at about eight minutes. At this time, about 7 ml of solution had been delivered. The approximate extent of the solution at this time is illustrated in FIG.21(a).

The second line 720 (corresponding to the output of the second interior sensor 512) intersects the threshold line 760 at point 721, which indicates that the second interior sensor 512 was triggered at about thirteen minutes. At this time, about 12 ml of solution had been delivered. The approximate extent of the solution at this time is illustrated in FIG.21(b).

The third line 730 (corresponding to the output of the third interior sensor 513) intersects the threshold line 760 at point 731, which indicates that the third interior sensor 513 was triggered at about 32 minutes. At this time, about 26 ml of solution had been delivered. The approximate extent of the solution at this time is illustrated in FIG.21(c).

The fourth line 740 (corresponding to the output of the first border sensor 521) intersects the threshold line 760 at point 741, which indicates that the first border sensor 521 was triggered at about 80 minutes. At this time, about 67 ml of solution had been delivered. The approximate extent of the solution at this time is illustrated in FIG.21(d).

The fifth line 750 (corresponding to the output of the second border sensor 522) intersects the threshold line 760 at point 751, which indicates that the second border sensor 522 was triggered at about 93 minutes. At this time, about 78 ml of solution had been delivered. The approximate extent of the solution at this time is illustrated in FIG.21(e).

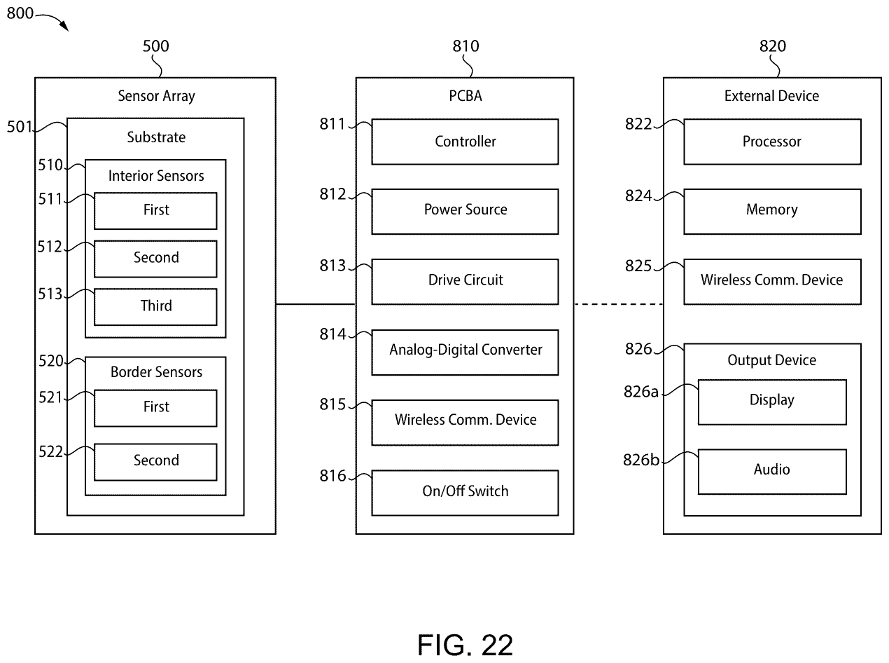

With additional reference to FIG. 22, illustrated therein is a schematic block diagram of a system 800 according to certain embodiments. The system 800 generally includes the sensor array 500, a printed circuit board assembly (PCBA) 810 connected with the sensor array 500, and an external device 820 in communication with the PCBA 810. As will be appreciated, the sensor array 500 and PCBA 810 may be provided in a wound dressing such as the wound dressing 100. For example, the sensor array 500 may be utilized as the sensor array 152 and the PCBA 810 may be utilized as the PCBA 154.

The PCBA 810 is in electrical communication with the sensor array 500, and in the illustrated form includes a controller 811, a power source 812 such as a coin cell battery, a drive circuit 813 such as one of the above-described drive circuits, an analog-digital converter (ADC) 814, a communication device such as a wireless communication device 815, and an on/off switch 816.

The external device 820 is in communication with the PCBA 810, and generally includes a processor 822, a memory 824, a communication device such as a wireless communication device 825, and an output device 826, such as a display 826a and/or an audio output device 826b. In certain embodiments, the external device 820 is a mobile computing device, such as a smartphone or tablet. In certain embodiments, the external computing device 820 is positioned remote from the wound dressing 100, such as a location at least one foot from the wound dressing. The communication device 825 facilitates communication (e.g., wireless communication) with the PCBA 810. In certain embodiments, the communication device 825 may include a Bluetooth communication device, such as a Bluetooth Low Energy communication device. The external device 820 receives data from the PCBA 810 and performs one or more actions, such as generating alerts, based upon the data. For example, the external device 820 may generate a notification that the wound dressing 100 is in need of a change in response to the data generated by one or both of the corner sensors 521, 522 indicating that fluid (e.g., exudate) has reached the corner regions 504 of the dressing.

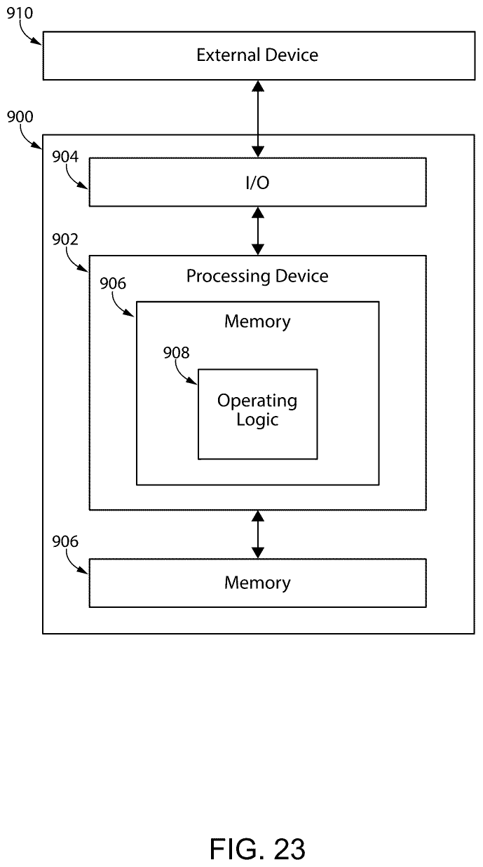

Referring now to FIG. 23, a simplified block diagram of at least one embodiment of a computing device 900 is shown. The illustrative computing device 900 depicts at least one embodiment of a computing that may be utilized in connection with the PCBA 810 and external device 820 illustrated in FIG. 22.

Depending on the particular embodiment, the computing device 900 may be embodied as a server, desktop computer, laptop computer, tablet computer, notebook, netbook, Ultrabook™ mobile computing device, cellular phone, smartphone, wearable computing device, personal digital assistant, Internet of Things (IoT) device, control panel, processing system, router, gateway, and/or any other computing, processing, and/or communication device capable of performing the functions described herein.

The computing device 900 includes a processing device 902 that executes algorithms and/or processes data in accordance with operating logic 908, an input/output device 904 that enables communication between the computing device 900 and one or more external devices 910, and memory 906 which stores, for example, data received from the external device 910 via the input/output device 904.

The input/output device 904 allows the computing device 900 to communicate with the external device 910. For example, the input/output device 904 may include a transceiver, a network adapter, a network card, an interface, one or more communication ports (e.g., a USB port, serial port, parallel port, an analog port, a digital port, VGA, DVI, HDMI, Fire Wire, CAT 5, or any other type of communication port or interface), and/or other communication circuitry. Communication circuitry may be configured to use any one or more communication technologies (e.g., wireless or wired communications) and associated protocols (e.g., Ethernet, Bluetooth®, Bluetooth Low Energy (BLE), Wi-Fi®, WiMAX, etc.) to effect such communication depending on the particular computing device 900. The input/output device 904 may include hardware, software, and/or firmware suitable for performing the techniques described herein.

The external device 910 may be any type of device that allows data to be inputted or outputted from the computing device 900. For example, in various embodiments, the external device 910 may be embodied as the sensor array 500, the PCBA 810, the external device 820, or an electronic component of the sensor array 500, the PCBA 810, or the external device 820. Further, in some embodiments, the external device 910 may be embodied as another computing device, switch, diagnostic tool, controller, printer, display, alarm, peripheral device (e.g., keyboard, mouse, touch screen display, etc.), and/or any other computing, processing, and/or communication device capable of performing the functions described herein. Furthermore, in some embodiments, it should be appreciated that the external device 910 may be integrated into the computing device 900.

The processing device 902 may be embodied as any type of processor(s) capable of performing the functions described herein. In particular, the processing device 902 may be embodied as one or more single or multi-core processors, microcontrollers, or other processor or processing/controlling circuits. For example, in some embodiments, the processing device 902 may include or be embodied as an arithmetic logic unit (ALU), central processing unit (CPU), digital signal processor (DSP), and/or another suitable processor(s). The processing device 902 may be a programmable type, a dedicated hardwired state machine, or a combination thereof. Processing devices 902 with multiple processing units may utilize distributed, pipelined, and/or parallel processing in various embodiments. Further, the processing device 902 may be dedicated to performance of just the operations described herein, or may be utilized in one or more additional applications. In the illustrative embodiment, the processing device 902 is of a programmable variety that executes algorithms and/or processes data in accordance with operating logic 908 as defined by programming instructions (such as software or firmware) stored in memory 906. Additionally or alternatively, the operating logic 908 for processing device 902 may be at least partially defined by hardwired logic or other hardware. Further, the processing device 902 may include one or more components of any type suitable to process the signals received from input/output device 904 or from other components or devices and to provide desired output signals. Such components may include digital circuitry, analog circuitry, or a combination thereof.

The memory 906 may be of one or more types of non-transitory computer-readable media, such as a solid-state memory, electromagnetic memory, optical memory, or a combination thereof. Furthermore, the memory 906 may be volatile and/or nonvolatile and, in some embodiments, some or all of the memory 906 may be of a portable variety, such as a disk, tape, memory stick, cartridge, and/or other suitable portable memory. In operation, the memory 906 may store various data and software used during operation of the computing device 900 such as operating systems, applications, programs, libraries, and drivers. It should be appreciated that the memory 906 may store data that is manipulated by the operating logic 908 of processing device 902, such as, for example, data representative of signals received from and/or sent to the input/output device 904 in addition to or in lieu of storing programming instructions defining operating logic 908. As illustrated, the memory 906 may be included with the processing device 902 and/or coupled to the processing device 902 depending on the particular embodiment. For example, in some embodiments, the processing device 902, the memory 906, and/or other components of the computing device 900 may form a portion of a system-on-a-chip (SoC) and be incorporated on a single integrated circuit chip.

In some embodiments, various components of the computing device 900 (e.g., the processing device 902 and the memory 906) may be communicatively coupled via an input/output subsystem, which may be embodied as circuitry and/or components to facilitate input/output operations with the processing device 902, the memory 906, and other components of the computing device 900. For example, the input/output subsystem may be embodied as, or otherwise include, memory controller hubs, input/output control hubs, firmware devices, communication links (e.g., point-to-point links, bus links, wires, cables, light guides, printed circuit board traces, etc.) and/or other components and subsystems to facilitate the input/output operations.

The computing device 900 may include other or additional components, such as those commonly found in a typical computing device (e.g., various input/output devices and/or other components), in other embodiments. It should be further appreciated that one or more of the components of the computing device 900 described herein may be distributed across multiple computing devices. In other words, the techniques described herein may be employed by a computing system that includes one or more computing devices. Additionally, although only a single processing device 902, I/O device 904, and memory 906 are illustratively shown in FIG. 23, it should be appreciated that a particular computing device 900 may include multiple processing devices 902, I/O devices 904, and/or memories 906 in other embodiments. Further, in some embodiments, more than one external device 910 may be in communication with the computing device 900.

As noted above, the sensor layout illustrated in FIG. 16 is one potential configuration for a sensor array. Those skilled in the art will readily appreciate that the measurement process described herein may take into consideration the electrochemical side-effects, safety requirements, and optimization of power consumption. For example, the measurement system may be configured to adhere to one or more of the following guidelines. In certain forms, the system may limit bias voltages to a threshold minimum in order to avoid redox reactions between the electrodes and the electrolyte. In certain forms, the system may limit bias pulse timing to very short pulses, in the single digit millisecond range, to reduce power consumption, limit redox reactions and avoid 50 Hz or 60 Hz noise interference. In certain forms, the system may achieve a full AC bias waveform, with DC balanced to zero. In certain forms, the system may consider that the ADC inputs have high impedance and may be subject to ambient noise. Additionally or alternatively, the series resistors may be selected to be compliant to 60601 Electrical Safety requirement for body worn devices.

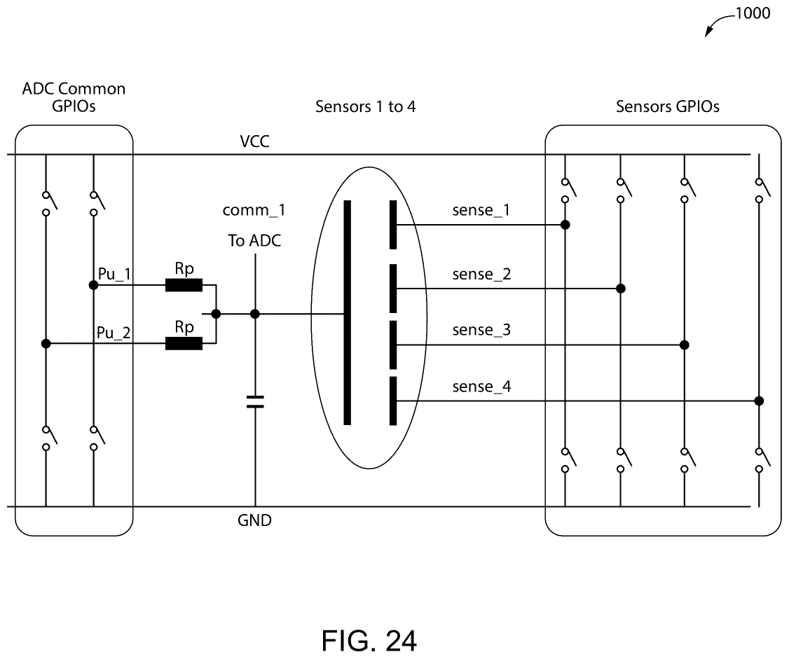

With additional reference to FIG. 24, illustrated therein is an example control circuit 1000 for controlling a sensor array with N sensors. In the illustrated form, the number N is four. However, it should be appreciated that the control circuit 1000 may be modified to account for more or fewer sensors. In the control circuit 1000, a single ADC line comm_1 is used to measure the conductivity of the N sensors. One sensor at a time is driven with a specific bias cycle, while the others are kept isolated, by putting the dedicated side of the sensor (sensor_x) in three state. As soon as there is a small conductivity among the tracks, the GPIO lines left in high impedance will follow the signals from other GPIOs or comm_1 line, resulting in a removal of any DC component. The driving waveform is based on a ½ VCC step at a time on the comm_1 electrode side and a direct/reverse polarization on the sensor_x electrode side. This may allow for the creation of a positive and negative bias on the sensor and a full symmetrical waveform in voltage and timing.

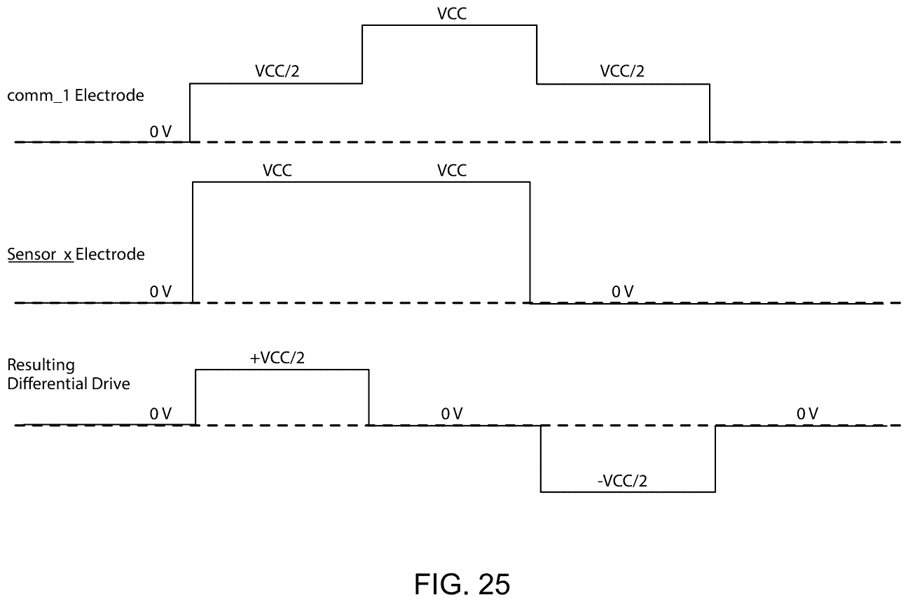

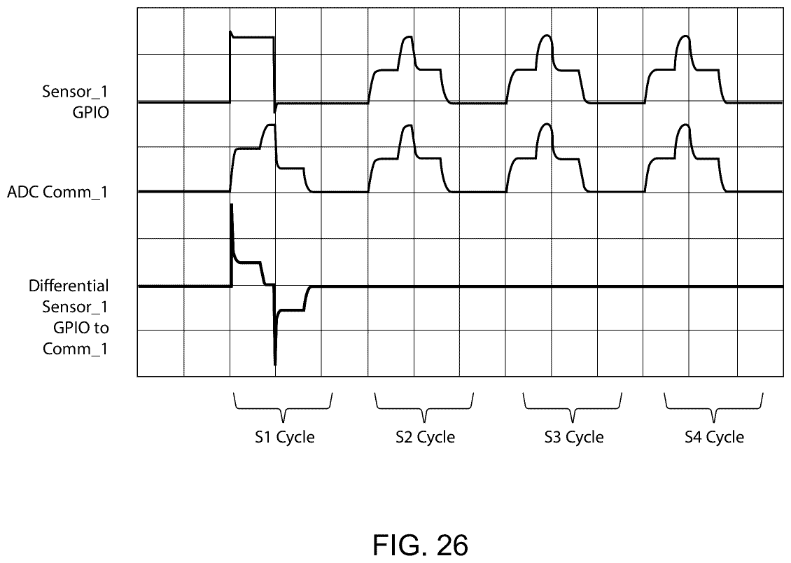

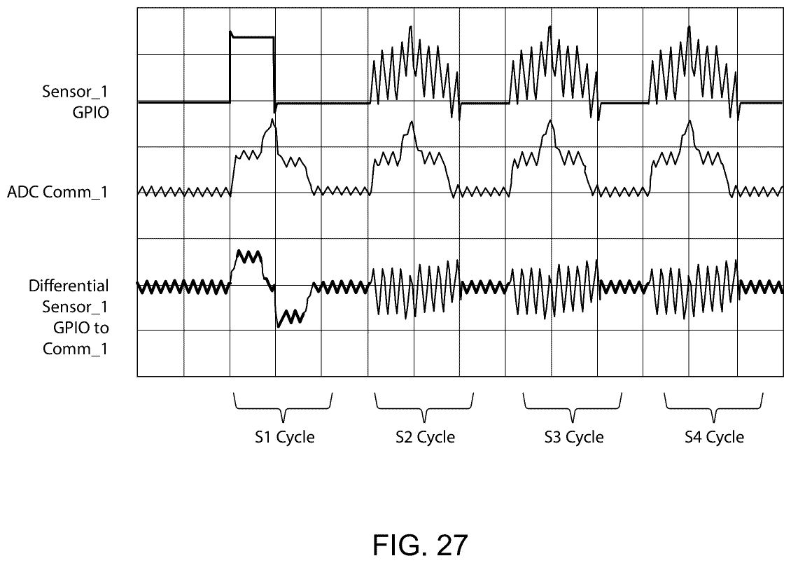

With additional reference to FIGS. 25-27, illustrated therein are graphs illustrating operation of the control circuit 1000. In FIG. 26, the line of sensor_1 is driven in Sensor1 cycle, while it is left floating when sensor_2, sensor_3, or sensor_4 is measured. In the test, sensor_1 is mildly wet. The differential signal is present only when sensor_1 is biased for testing, while the sensor_1 line copies the common line while the other three sensors are measured, resulting in a flat line as a differential voltage. FIG. 27 is substantially similar to FIG. 26, but a 1 KHz noise has been imposed to the sensor tracks to highlight the high impedance of the GPIO Sensor_1.

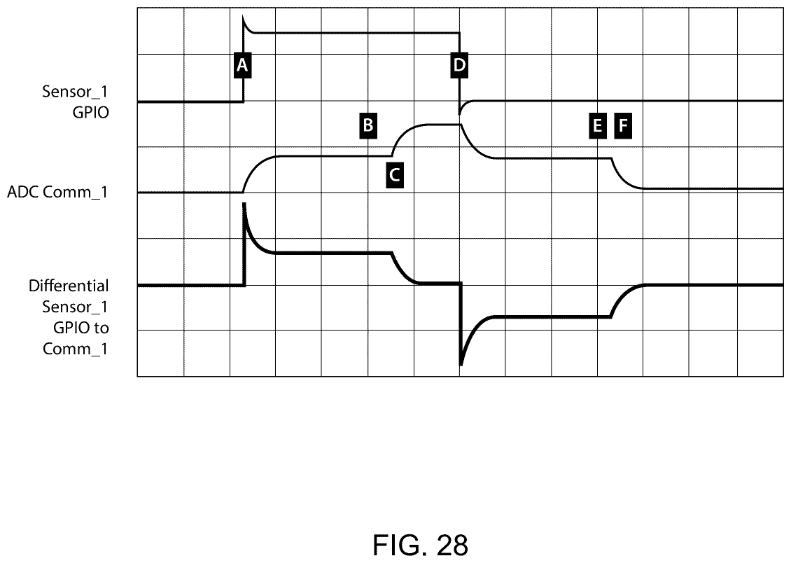

With additional reference to FIG. 28, illustrated therein is an example bias sequence for one sensor of the control circuit 1000. In a starting configuration, pu_1 is LOW, pu_2 is LOW, sensor_1 is LOW, and the remaining sensors are in the Three State. At Point A, the GPIO of sensor_1 is driven HIGH, and pu_1 is driven HIGH; the target voltage is VCC/2. At Point B, the ADC samples the voltage; if there is conductivity between sensor_1 and comm_1, point B will be higher than VCC/2. At Point C, pu_2 is driven HIGH, and the target voltage is VCC. At Point D, the GPIO of sensor_1 is driven LOW, pu_2 is LOW, and the target voltage is VCC/2. At Point E, the ADC samples the voltage; if there is conductivity between sensor_1 and comm_1, point E will be lower than VCC/2. At Point F, pu_1 is LOW, and the target voltage is GND. In an ending configuration, pu_1 is LOW, pu_2 is LOW, and all sensors are set to LOW for sleep mode. With this sequence, the differential voltage seen by two tracks of the sensor is only AC and has a fully symmetrical waveform. For signal computation, the data is computed as the difference between Voltage (B) and Voltage (E).

In certain embodiments, the ADC input may have a low pass filter for antialiasing and noise suppression. The capacitor can have an effect on the RC timing of the pu_1 and pu_2 GPIO switching. With Rp set to 23 K (and C=10 nF, the RC constant is 230 μs, rounded to 250 μs for tolerances. In certain embodiments, sampling Point B and sampling point E may occur later than 5*RC, (e.g., 1.25 ms), from pu_x line change. In certain embodiments, point C and point F may take place after ADC “sample and hold” is completed. In certain embodiments, the “sample and hold” time period may be about 40 μs (e.g., 30 μs to 50 μs), or about 100 μs (e.g., 80 μs to 120 μs). The final measurement forwarded out from the sampling code may be shared to main code in plural variables, such as one per each sensor. In certain embodiments, these variables may be represented as an eight-bit integer showing the ADC count difference.

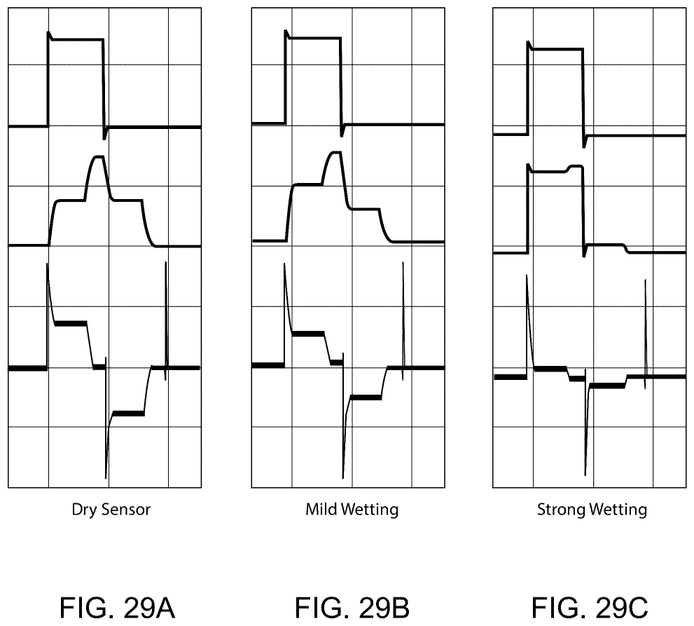

With additional reference to FIG. 29A-29C, illustrated therein are examples of behavior at different wetting conditions. More particularly, FIG. 29A illustrates output with a dry sensor, FIG. 29B illustrates output with a mildly wet sensor, and FIG. 29C illustrates the output with a strong wetting. As will be evident from inspection of the graphs, higher voltage difference is present when the sensor tracks are well wet.

Due to the presence of RC networks and an AC biasing scheme, the time sequencing of the GPIO updates and ADC sampling points should be assured. More particularly, no interrupts should occur, as such interrupts could separate the two actions by inserting additional delays. In certain forms, a full hardware driven solution is suggested in controlling GPIOs and ADC. The higher level tasks can be driven by software timers.

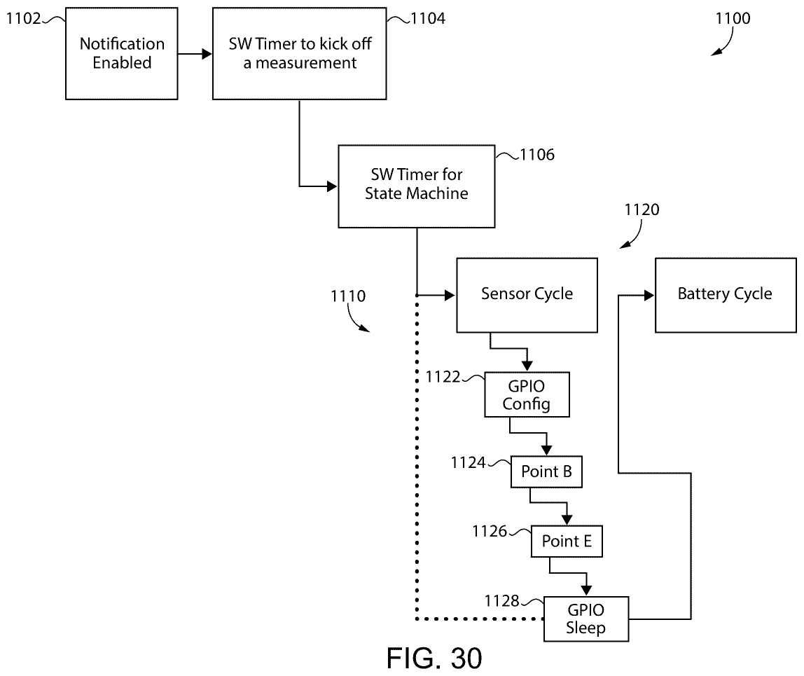

With additional reference to FIG. 30, illustrated therein is a schematic flowchart of a process 1100 according to certain embodiments. The process 1100 may begin with the enabling of a notification at block 1102. For example, block 1102 may occur when BLE notifications are enabled and a new set of data should be provided. In response to the enabling of notifications, the process 1100 may proceed to block 1104, which generally involves initiation of a timer to begin measurement. The timer initiated in block 1104 may periodically initiate a second timer in block 1106 to run the measurement system, for example via a state machine. The state machine may be non-blocking, and may run a sequence 1110 to perform measurements and enter a battery voltage measurement.

The sequence 1110 may include a sensor cycle 1120 that begins with a selected first sensor. In block 1122, the GPIO configuration is run as described above. In block 1124, the voltage at the time of Point B is determined. In block 1126, the voltage at the time of Point E is determined. In block 1128, the GPIO is put to sleep. The sensor cycle procedure 1120 may then be repeated for each of the remaining sensors, as indicated by the dashed line. Once the sensor cycle 1120 has been performed for all sensors (or at least one or more selected sensors), the sequence 1110 may proceed to block 1112, which generally involves measuring battery voltage. Each sensor cycle 1120 may require GPIO configuration and the set up and triggering of hardware assisted functions via hardware timers. Due to the limitations of hardware functions, the sampling process may be divided in half, such that the first half controls GPIO and ADC to collect the voltage in point B of the bias cycle, while the second half drives the GPIO and ADC for point E. At the end of the cycle 1120, GPIOs may be restored to a default condition to enter a sleep mode.

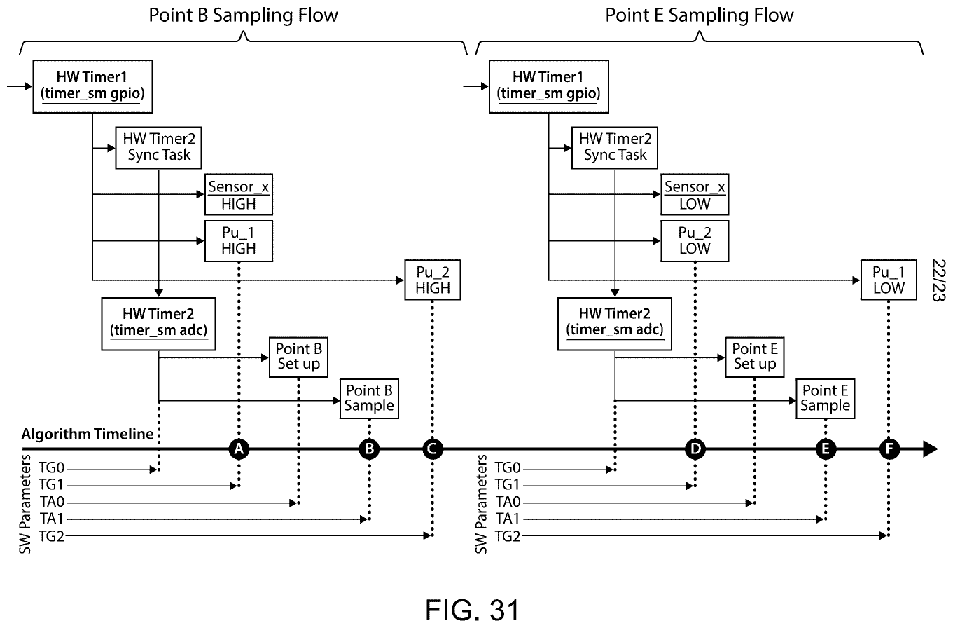

With additional reference to FIG. 31, further details regarding the control of the control circuit 1000 will now be provided. In certain embodiments, the sequencing of the GPIO states and ADC sampling is fully executed by a hardware machine, configured in the device by software, at run time. There are two sampling points, as described previously, referenced as Point B and Point E. Each point has a dedicated configuration which prepares and runs two timers in parallel and specific GPIO control tasks, which may be hardware assisted. For the GPIO control hardware machine, one timer timer_sm_gpio (e.g., a hardware timer) may manage the hardware control of the GPIOs of the sensor lines, sensor_x and the pu_1 and pu_2 lines. A second timer timer_sm_adc, in sync with the previous one, set up and then fires the ADC to take the sample.

One or more events that fire hardware tasks may be scheduled via capture-compare thresholds. In certain embodiments, the ADC timer is hardware synchronized by a hardware event from the GPIO timer to avoid jitter effects from interrupt requests (IRQs). The software state machine may program the HW blocks separately for the two cycles, for point B and point E, for example due to hardware limitations of the resources. Note also that the two cycles may be unequal, since pu_1 GPIO and pu_2 GPIO actions are not equivalent or symmetrical in time. Synchronization of hardware events and hardware tasks may, for example, be accomplished using a parallel peripheral interface (PPI), which connects timer capture-compare events to hardware events (e.g., GPIO changes and ADC start). To properly trigger the ADC with PPI and keep the low power current consumption, one or both of two events may be performed: a first to first set the ADC, and a second to fire the sampling. To change the status of sensor_x GPIO and the pu_x GPIO at the same time, a PPI fork configuration may be utilized, such that the two tasks are connected to the same capture-compare event.

As noted above, it may be warranted to account for safety requirements for patient auxiliary current injected into the body during the measurement. The system may be battery operated and/or considered an electrically floating device. The measurement may adopt an AC driving scheme. In certain embodiments, the safety criteria applied are a 100 μA max in normal conditions, and a 500 μA max in single fault condition. The maximum current that can be injected into the patient in normal conditions is limited by the Rp series resistors. In case of a 47 kΩ value and a battery voltage of 3.2 V, the max differential voltage is 1.6 V with a parallel Rp of 23.5 kΩ, leading to a max current of 68 μA. This is the worst case, assuming a zero ohm body resistance, zero ohm trace resistance, and zero ohm internal battery resistance.

Two single fault scenarios are considered in detail (as the others may be symmetrical). First, one of the two pull up resistors may be faulty and in a condition of short circuit, with zero resistance. Second, one of the GPIOs driving the sensor_x line may be faulty and driving the line to ground voltage with low impedance. Firmware may implement a safety check to detect these two failure modes. In certain forms, the sense_1, sense_2, sense_3, and sense_4 GPIOs are left in three state, while the GPIO driving Pu_1 is set to VCC and GPIO driving Pu_2 is set to GND. In absence of a fault, the comm_1 line going to the ADC is aligned to VCC/2. The comm_1 voltage is compared against a bandgap of VCC/2+/−20%. If the test is passed, the system is allowed to perform a measurement driving the sense_x GPIO.

Detection of fault type A (failure of a pull-up resistor) may be achieved along the following lines. In case one of the two Rp resistors is shorted, the voltage will become close to VCC or to GND and the fault will be detected by the safety test. It is also contemplated that the Rp resistor may be split into two devices, so that even in case of a single point of failure in a short condition, the other resistor is still operational. In this way, the hardware will be limiting the current, in single fault of Rp, to a threshold value (e.g., 136 μA).

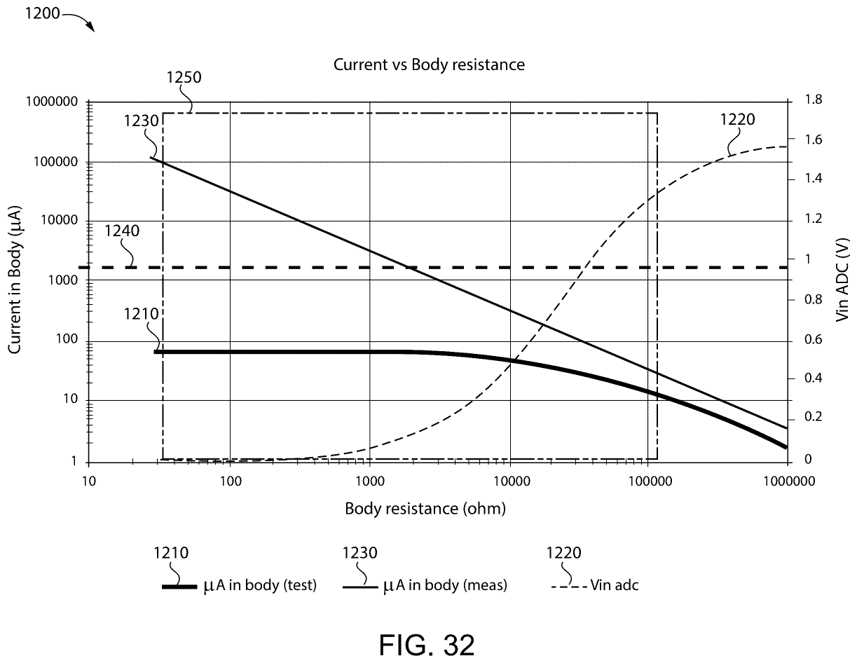

With additional reference to FIG. 32, detection of fault type B (failure of GPIO) may be achieved along the following lines. Note that the worst case scenario may occur when this fault is present and one of the other sense_x lines is driven to VCC during the measurement. As a result, it may be warranted to detect the GPIO failure before driving the other lines. FIG. 31 is a graph 1200 illustrating selected electrical characteristics during performance of a test. Line 1210 shows the current injected into the body when the safety test is performed, depending on the body skin resistance. Line 1220 shows the voltage measured by the safety system, and line 1222 shows the threshold for this voltage. Line 1230 shows the current that will be injected into the body in case of single GPIO fault during the measurement. Line 1240 shows the maximum allowed current in a single fault condition. The area in rectangle 1250 is the detectability zone. In case of single fault on GPIO, the safety test process will inject up to 70 μA maximum. The maximum injected current in case of a non-detected GPIO failure is less than 30 μA maximum.

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected.

It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

What is claimed is:

1. A wound dressing, comprising:
a wound-contacting layer; and
a sensor array, comprising:
a substrate;
a first sensor positioned on the substrate; and
a second sensor positioned on the substrate;
wherein the second sensor surrounds the first sensor; and
wherein each of the first sensor and the second sensor has a corresponding and respective electrical characteristic that varies in response to contact with wound exudate.

2. The wound dressing of claim 1, wherein the substrate is transparent.

3. The wound dressing of claim 1, wherein the electrical characteristic comprises impedance.

4. The wound dressing of claim 1, further comprising a wireless communication device configured to transmit information generated by the sensor array to an external device.

5. The wound dressing of claim 4, wherein the wireless communication device comprises a Bluetooth communication device.

6. The wound dressing of claim 1, wherein each of the first sensor and the second sensor comprises a corresponding and respective trace on the substrate.

7. The wound dressing of claim 6, wherein each trace comprises an ink comprising silver.

8. The wound dressing of claim 6, further comprising at least one protective layer covering a portion of each trace and isolating the portion from contact with exudate.

9. The wound dressing of claim 1, wherein each of the first sensor and the second sensor is positioned within an interior region of the substrate; and
wherein the sensor array further comprises a third sensor positioned within a border region of the substrate.

10. The wound dressing of claim 9, wherein the third sensor includes a first sensing region positioned in a first corner region of the substrate.

11. The wound dressing of claim 10, wherein the third sensor further includes a second sensing region positioned in a second corner region of the substrate.

12. The wound dressing of claim 11, wherein the third sensor further comprises a non-sensing region positioned between the first sensing region and the second sensing region; and
wherein the non-sensing region is isolated from contact with exudate by a protective layer.

13. The wound dressing of claim 1, wherein the first sensor is positioned within an interior region of the substrate; and
wherein the second sensor is positioned within a border region of the substrate.

14. The wound dressing of claim 1, further comprising a printed circuit board assembly configured to facilitate control of the sensor array;
wherein each of the first sensor and the second sensor is connected with the printed circuit board assembly via anisotropic conductive film.

15. The wound dressing of claim 14, wherein the printed circuit board assembly comprises a wireless communication device.

16. The wound dressing of claim 1, further comprising:
a foam layer positioned between the wound-contacting layer and the sensor array; and
an absorbent layer positioned between the foam layer and the sensor array.

17. A method, comprising:
receiving information generated by a plurality of sensors of a wound dressing;
comparing the information to at least one threshold;
determining, based upon the comparing, (a) a wetness of the wound dressing, and/or (b) a recommendation regarding changing of the wound dressing; and
generating an output based on the determining, wherein the output relates to (a) the wetness of the wound dressing, and/or (b) the recommendation regarding changing of the wound dressing.

18. The method of claim 17, wherein each of the receiving, the comparing, the determining, and the generating is performed by one or more computing devices positioned remotely from the wound dressing.

19. The method of claim 17, wherein the receiving comprises receiving the information via a wireless communication connection.

20. The method of claim 19, wherein the method is performed by a mobile device.

21. The method of claim 19, wherein the wireless communication connection comprises a Bluetooth connection.

22. The method of claim 17, wherein the information comprises first information generated by a first sensor of the plurality of sensors, and second information generated by a second sensor of the plurality of sensors;
wherein comparing the information to at least one threshold comprises comparing the first information to a first threshold, and comparing the second information to a second threshold;
wherein determining (a) the wetness of the wound dressing, and/or (b) the recommendation regarding changing of the wound dressing comprises:
determining whether a first zone of the wound dressing is wet based upon the comparing of the first information to the first threshold; and
determining whether a second zone of the wound dressing is wet based upon the comparing of the second information to the second threshold; and
wherein the output relates to wetness of the first zone and/or wetness of the second zone.

23. The method of claim 22, wherein the first threshold and the second threshold are equal to one another.

24. The method of claim 17, wherein generating the output comprises displaying a message on a display.

25. The method of claim 24, wherein the display is a display of a mobile device.

26. The method of claim 17, wherein generating the output comprises generating an audible alert.

27. The method of claim 17, wherein the receiving, the comparing, the determining, and the generating are performed by at least one first computing device; and
wherein generating the output comprises transmitting the output to at least one second computing device.

28. A non-transitory computer readable medium comprising instructions that, when executed by a processor of a computing device, cause the computing device to perform the method of claim 17.

29. A mobile device comprising a processor and a non-transitory computer readable medium comprising instructions that, when executed by the processor, cause the mobile device to perform the method of claim 17.

30. An apparatus, comprising:
a wound dressing comprising:
a wound-contacting layer; and
a sensor array positioned distally of the wound-contacting layer, the sensor array comprising a first impedance sensor; and
a printed circuit board assembly (PCBA) in electrical communication with the sensor array, the PCBA including a wireless communication device operable to wirelessly transmit information generated by the sensor array.

31. The apparatus of claim 30, wherein the sensor array further comprises a second impedance sensor.

32. The apparatus of claim 31, wherein the first impedance sensor is nested within the second impedance sensor.

33. The apparatus of claim 32, wherein the first impedance sensor is configured to detect exudate within an interior region of the wound dressing; and
wherein the second impedance sensor is configured to detect exudate within a border region of the wound dressing.

34. The apparatus of claim 30, wherein the first impedance sensor is configured to detect exudate in a corner region of the wound dressing.

35. The apparatus of claim 30, wherein the PCBA is in electrical communication with the sensor array via an anisotropic conductive film.

36. The apparatus of claim 30, wherein the sensor array further comprises a substrate; and
wherein the first impedance sensor comprises a trace printed on the substrate.

37. The apparatus of claim 36, wherein the first impedance sensor comprises an ink comprising silver.

38. The apparatus of claim 30, wherein a portion of the trace is covered by a protective layer that protects the portion of the trace from contact with exudate.

39. The apparatus of claim 30, wherein the PCBA is configured to apply an alternating current to the sensor array, to determine an impedance of the first impedance sensor, and to transmit to an external device information related to the impedance of the first impedance sensor.

40. A wound dressing, comprising:
a wound-contacting layer; and
a sensor array positioned distally of the wound-contacting layer, the sensor array comprising:
a substrate;
a plurality of interior traces printed on an interior region of the substrate, the plurality of interior traces comprising a first interior trace and a second interior trace nested within the first interior trace; and
at least one border trace printed on a border region of the substrate;
wherein each interior trace and each border trace has a corresponding and respective impedance that varies in response to contact with exudate.
